(19) United States
(12) Patent Application Publication
(10) Pub. No.: US 2010/0324546 A1
(43) Pub. Date: Dec. 23, 2010

(54) CRYOSHEATH

(76) Inventors: Alexander Levin, Binyamina (IL); Didier Toubia, Ra'anana (IL); Miron Kaganovich, Haifa (IL)

Correspondence Address: DR. D. GRAESER LTD., 9003 FLORIN WAY, UPPER MARLBORO, MD, 20772, US

(21) Appl. No.: 12/668,428

(22) PCT Filed: Jul. 9, 2008
(86) PCT No.: PCT/IL2008/000943

§ 371 Date: Sep. 7, 2010

Related U.S. Application Data
(60) Provisional application No. 60/948,502, filed on Jul. 9, 2007.

(51) Int. Cl. A61B 18/14

(52) U.S. Cl. 606/24

(57) ABSTRACT

A device, system and method for a cryoprobe which features a heating component for heating at least a proximal portion of the tip of the cryoprobe, thereby preventing damage to an adjacent portion of tissue.

### FIELD OF THE INVENTION

The present invention relates to the field of cryosurgical technique and, especially, to cryoprobes intended to perform cryoablation of tissue upon immediate contact with their cryotips.

### BACKGROUND OF THE INVENTION

It is known that most cryoprobes in common use today operate on the basis of the Joule-Thomson effect or by boiling a liquid cryogen supplied on the internal surface of the cryoprobe tip. As a rule, the length of the cryotip (the tip of the cryoprobe) is significantly larger than its diameter. The low cryogenic temperature of the cryotip causes the temperature of the cryoprobe shaft in the immediate vicinity of the cryotip to be lowered, because of the thermal conductivity of the body of the cryoprobe shaft. In some cases, the distance from the superficial layer of an organ until the distal point of the tumor to be ablated is smaller than the length of an ice-ball formed around the cryotip inserted into this tumor. This shorter distance can cause, in turn, damage of the superficial (upper or outer) layers of the organ at the entry location of the cryoprobe. For example, this problem can appear in the cryo-treatment of a superficial fibroadenoma of the breast.

Some US patents and patent applications describe disposable sheaths, which are positioned on a cryoprobe shaft and are intended to protect surrounding healthy tissue from damage due to excessive cold temperatures. However, they are not suitable for protecting the superficial layer of skin or other organs.

For example, US patent application No. 20080119838 describes a sheath for use with a cryoprobe in a cryosurgical procedure; this sheath can have an opening at its proximal end configured to be inserted over a tip portion of a cryoprobe. The sheath can be connected to the cryoprobe with an end connector. Sheath can have a tip located at its distal that can have a trocar configuration for tissue penetration. A cylindrical section located inwardly from the tip can have a spiral groove defined on an exterior surface. At least one wire can be disposed within the groove so as to fit within the outer perimeter of the sheath. Wires disposed in grooves can include heating elements and temperature sensing elements.

US patent application No. 20050038422 describes a cryosurgical apparatus, which includes an elongate cryoprobe having an electrically conductive first portion and multiple cooling elements. A removable sheath having an electrically conductive second portion is received on the cryoprobe with its electrically conductive second portion spaced from the electrically conductive first portion of the cryoprobe. Electrical insulation is interposed between the first portion and the second portion. In operation, cooling elements in the cryoprobe cool the tissue around a portion of the cryoprobe while electromagnetic energy traveling between the first portion and the second portion heats tissue adjacent to the cooled tissue. The cooling alters the path of the electromagnetic energy by changing the electrical conductivity of the tissue in the region of the cryoprobe.

US patent application No. 20040024391 teaches an apparatus and method for protecting the neurovascular bundle during cryoablation of tissues of the prostate by heating the vicinity of the neurovascular bundle while cooling pathological tissues of a prostate to cryoablation temperatures, thereby cryoablating pathological tissues while protecting the neurovascular bundle from damage. A cryoprobe operable to cool a distal operating tip while heating a proximal shaft is presented. US patent application No. 20020022832 describes a cryoprobe assembly, which includes a cryoprobe and an outer sheath assembly detachably connected thereto. The cryoprobe includes: a Joule-Thomson nozzle; a high pressure gas supply line for supplying gas to the Joule-Thomson nozzle; a heat exchanger interposed between the high pressure gas supply line and the Joule-Thomson nozzle; a cryoprobe sheath containing the heat exchanger and Joule-Thomson nozzle, the cryoprobe sheath having an outer surface; and, a handle attached to the first sheath, the handle having a gripping portion directly graspable by an operator and a connecting portion. The outer sheath assembly includes an adapter covering substantially none of the gripping portion of the cryoprobe handle. The adapter is for attachment to the connecting portion. An outer sheath is connected to the adapter and surrounds the outer surface of the cryoprobe sheath wherein the outer sheath provides enhanced protection against any gas leaks.

U.S. Pat. No. 6,936,045 describes a malleable cryosurgical probe that includes a cryostat assembly and a cryoprobe assembly. The cryostat assembly includes an elongated shaft assembly having at least one malleable segment thereof and a closed distal end. The shaft assembly includes at least one freezing portion, at least one thermally insulated portion and a thermally insulating element positioned about the thermally insulated portion.

U.S. Pat. No. 5,720,743 teaches a surgical probe having the ability to thermally insulate adjacent healthy tissue from cooling or heating effects at adjacent tissue being subjected to thermal destruction by cryosurgery, electrosurgery and hyperthermia. The probe includes a spatula shaped thermally insulating member that is mounted at the distal end of an elongated rigid member of a width sized for insertion through a small cutaneous incision. The insulating spatula is of a material transparent to ultrasound thus allowing ultrasonic imaging of tissue through the thermal insulator without substantial image deterioration.

Unfortunately, none of the above solutions is suitable for warming the superficial layers of the skin or other organs.

### SUMMARY OF THE INVENTION

The background art does not teach or suggest a cryoprobe which is able to treat lower tissue layers while preventing damage to superficial tissue layers or to surrounding tissue.

The present invention overcomes the drawbacks of the background art by providing a device, system and method for a cryoprobe which features a heating component for heating at least a proximal portion of the tip of the cryoprobe, thereby preventing damage to an adjacent portion of tissue, preferably for superficial layers of the skin or other organs.

According to some embodiments of the present invention, there is provided a cryoprobe for superficial cryosurgical treatment, which permits freezing tumors located in an immediate vicinity to the superficial layers of an organ, and, at the same time, to preserve the superficial layers themselves from damage by cryoablation.

In order to achieve this, in at least some embodiments, the cryoprobe is preferably provided with a displaceable sheath; the distal edge of this displaceable sheath is preferably in immediate contact with the skin or situated in the vicinity of the skin. The displaceable sheath has means for warming the metal shaft of the cryoprobe and also optionally and more preferably the proximal section of the cryotip.

Such warming ensures a positive temperature of the portion(s) of the shaft or the cryotip in contact with the uppermost superficial skin layer; further, it also increases the resultant temperature gradient along the distal section of the cryoprobe shaft and/or the proximal section of the cryotip.

According to some embodiments of the present invention, the displaceable sheath has an internal diameter which is somewhat larger than the outer diameter of the cryoprobe; also, there is preferably a gap between the internal surface of the displaceable sheath and the external surface of the cryoprobe shaft, and/or the external surface of the cryoprobe shaft and the proximal section of the cryotip.

The internal surface of the sheath is preferably provided with at least one and more preferably a plurality of spacers, which ensures a uniform gap between the displaceable sheath and the cryoprobe shaft.

At its proximal edge, the displaceable sheath is slidingly positioned on the cryoprobe shaft, preferably with a close or tight fit. The proximal section of the displaceable sheath is provided with an inlet connection for supplying a warming gas into the gap between the cryoprobe shaft, the proximal section of the cryotip and the displaceable sheath. The distal edge of the displaceable sheath is optionally and preferably toothed; in such a way, the warming gas can be removed via this toothed edge to the atmosphere.

The sheath is preferably provided as well with a locking unit in order to fix the displaceable sheath in a certain position with respect to the cryoprobe shaft.

According to another preferred embodiment, a displaceable sheath is positioned with a close-sliding fit on the cryoprobe shaft. The displaceable sheath is preferably provided with an electrical heating coil, which can be applied as well for measuring the temperature of the displaceable sheath. As in the above embodiment, there is preferably a locking unit to fix the displaceable sheath in a certain position with respect to the cryoprobe shaft.

### BRIEF DESCRIPTION OF THE DRAWINGS

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice.

FIG. 1a and FIG. 1b show an exemplary embodiment of the present invention, with an axial cross-section of an exemplary cryoprobe with warming gas supplied into the gap between the cryoprobe shaft and the displaceable sheath; the displaceable sheath in these figures is positioned at the proximal and distal sections, respectively, of the cryoprobe.

FIG. 1c shows an enlarged section of the cryoprobe shaft with the displaceable sheath.

FIGS. 2a-2c show an axial cross-section of another exemplary cryoprobe with a displaceable sheath provided with an electrical heating coil.

### DESCRIPTION OF PREFERRED EMBODIMENTS

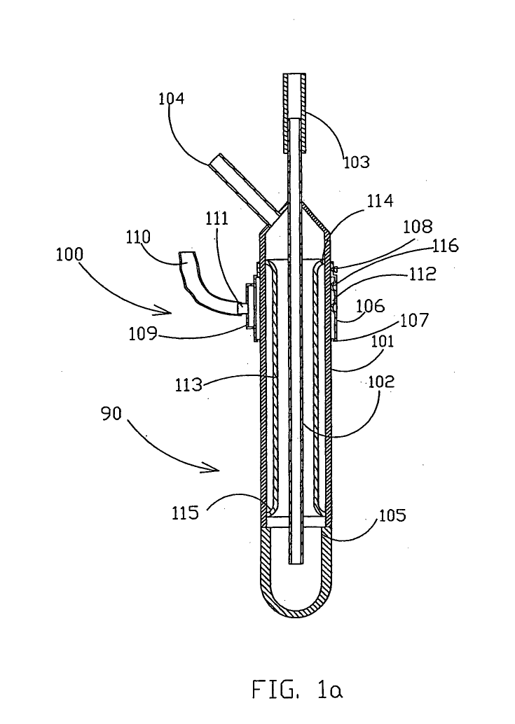

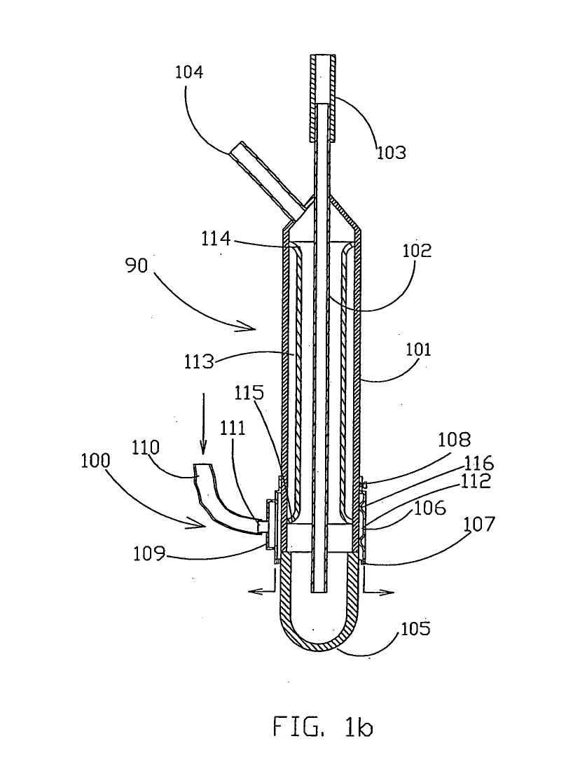

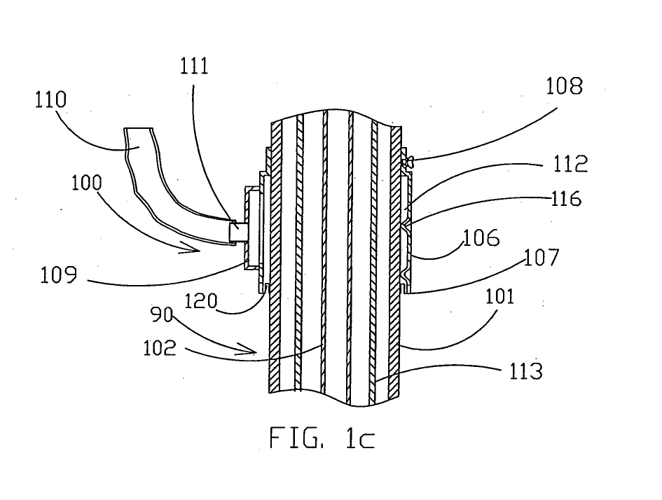

FIG. 1a and FIG. 1b show an exemplary embodiment of the present invention as an axial cross-section, showing the cryoprobe with a displaceable sheath which features a heating medium, preferably a heating gaseous medium, that is supplied into the gap between the cryoprobe shaft and the displaceable sheath. The displaceable sheath in these figures is positioned in a displaceable manner around the proximal and distal sections of the cryoprobe, such that the sheath moves up and down in a sliding manner as necessary.

In addition, FIG. 1c shows an enlarged section of the cryoprobe shaft with the displaceable sheath.

For all of FIGS. 1a-1c, a cryoprobe 90 comprises: a displaceable sheath 100; an external shaft 101; an internal feeding lumen 102, an inlet connection 103; an outlet connection 104; and cryotip 105. An intermediate tubular piece 113 with flanged edges 114 and 115 preferably serves for thermal insulation of a significant part of the external shaft 101.

The displaceable sheath 100 comprises envelope 106, which is positioned with a gap 112 around the external shaft 101 and the proximal section of the cryotip (in FIG. 1b). The distal edge of envelope 106 is toothed with one tooth 107 or optionally and preferably a plurality of teeth 107 in order to ensure free release of the warming gaseous medium when such teeth 107 are in immediate contact with the surface of a human organ (a skin or the external layer of an internal or external organ other than the skin). Teeth 107 are preferably cut into the distal edge of envelope 106 in such a manner that there is at least one gap between the teeth 107 for permitting release of the gaseous medium, while the distal edge of the teeth 107 preferably contacts the surface of a human organ. Optionally, a plurality of holes or other openings may also be provided at the distal edge of envelope 106, such that the distal edge of envelope 106 preferably contacts the organ while the holes or other openings permit the release of the gaseous medium.

Envelope 106 is provided with an opening 120 (or optionally openings), manifold 109 and an inlet connection 111 in manifold 109. Inlet connection 111 serves for entry of the heating gaseous medium from a supply conduit 110. In addition, envelope 106 is preferably provided with dimples (protrusions) 116 directed inwardly, which serve as spacers with respect to the external shaft 101 (although other configuration of the spacers may also optionally be considered).

Screw 108 at the distal section of envelope 106 secures the relative position of envelope 106 with respect to the external shaft 101.

The operation of cryoprobe 90 is as follows. Cryogen enters through inlet connection 103, for example from any type of cryogen supply source or device (such as a tank for example), to internal feeding lumen 102 and hence to cool cryotip 105. Providing the heating gaseous medium from supply conduit 110 enables the gaseous medium to enter inlet connection 111 in manifold 109, and hence to warm envelope 106, thereby warming external shaft 101. Preferably external shaft 101 is warmed substantially at the location of envelope 106, according to the position of displaceable sheath 100; the position of displaceable sheath 100 in turn is determined according to the depth of insertion of cryotip 105 into the skin or other organ.

Cryogen gas is exhausted through outlet connection 104, while the heating gaseous medium is exhausted through one or more openings 120.

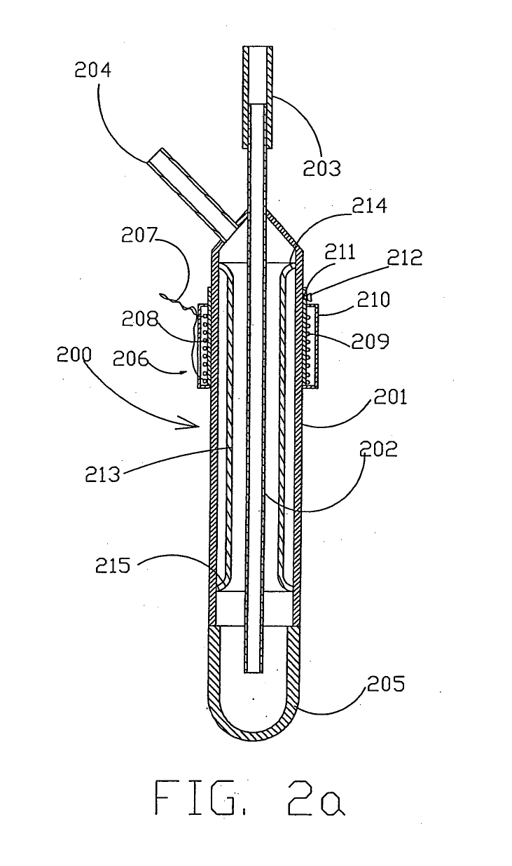

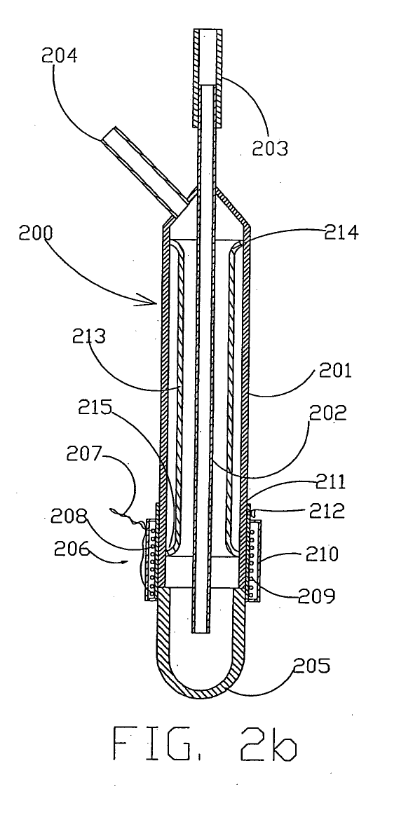

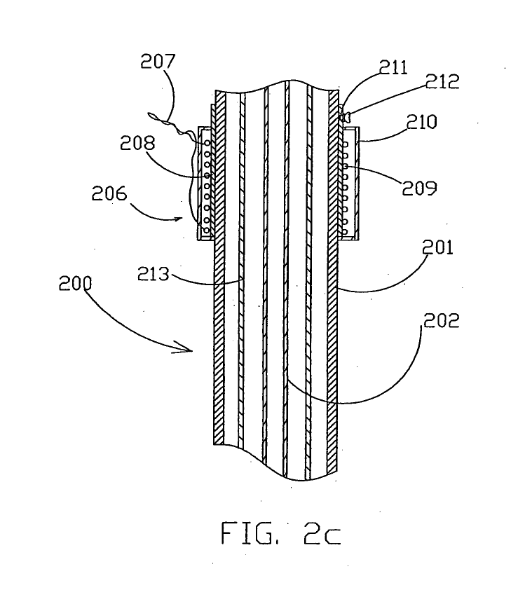

FIG. 2 shows an axial cross-section of another embodiment of the cryoprobe with a displaceable sheath provided with an electrical heating coil. A cryoprobe 200 comprises: an external shaft 201; an internal feeding lumen 202, an inlet connection 203; an outlet connection 204; and cryotip 205. An intermediate tubular piece 213 with flanged edges 214 and 215 preferably provides thermal insulation of at least a portion but more preferably a majority of the external shaft 101.

The displaceable sheath 206 comprises a sliding tubular piece 208 with an electrical heating coil 209, which is wound around or otherwise located adjacent to tubular piece 208; an electrical cable 207 provides energy to electrical heating coil 209 from a power source (not shown).

The proximal section of the sliding tubular piece 208 is provided with a threaded opening 211 and screw 212, for securing the position of the displaceable sheath 206 with respect to the external shaft 201. In such a way, the displaceable sheath 206 can be positioned on the external shaft 201 so that it covers a proximal section of cryotip 205, as illustrated in FIG. 2b.

An external cover 210 protects the electrical heating coil 209. The electrical heating coil 209 may also optionally be used to periodically measure electrical resistance thereby enabling the temperature of the sliding tubular piece 208 to be estimated and to regulate this temperature in a desirable range of temperatures. (Alternatively, a separate temperature sensor may be employed.) Resistance is generally linearly related to temperature and so can be easily correlated. Temperature control may then optionally be performed automatically, for example with some type of controller as is known in the art (not shown) by regulating the level of resistance.

The operation of cryoprobe 200 is similar to that described above with regard to the supply of cryogen. Cryogen enters through inlet connection 203, for example from any type of cryogen supply source or device (such as a tank for example), to internal feeding lumen 202 and hence to cool cryotip 205.

However, warming is now provided by electrical heating coil 209 upon supply of power, which warms tubular piece 208 and hence external shaft 201, preferably substantially at the location of tubular piece 208, according to the position of displaceable sheath 206; the position of displaceable sheath 206 in turn is determined according to the depth of insertion of cryotip 205 to the skin or other organ.

Cryogen gas is exhausted through outlet connection 204.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A cryoprobe operating with a cryogen comprising: an external shaft with an outlet connection for removal of gaseous cryogenic medium, said external shaft including an inner thermal insulation; an internal feeding lumen located within said external shaft, said internal feeding lumen serving to transmit the cryogen and having an inlet connection for receiving the cryogen; a cryotip joined with said external shaft and cooled with the cryogen transmitted by said internal feeding lumen; a displaceable sheath, which comprises an envelope positioned with a gap around said external shaft; and an inlet to said envelope for providing a warming gaseous medium into said gap.

2. The cryoprobe of claim 1, wherein said envelope comprises inward protrusions for providing a gap between said envelope and the external shaft.

3. A cryoprobe operating with a cryogen comprising: an external shaft with an outlet connection for removal of gaseous cryogenic medium, said external shaft including an inner thermal insulation; an internal feeding lumen located within said external shaft, said internal feeding lumen serving to transmit the cryogen and having an inlet connection for receiving the cryogen; a cryotip joined with said external shaft and cooled with the cryogen transmitted by said internal feeding lumen; and a displaceable sheath mounted adjacent said external shaft, the sheath comprising a sliding tubular piece with an electrical heating coil adjacent said tubular piece for warming said tubular piece.

4. The cryoprobe of claim 3, further comprising an electrical cable for energizing said electrical heating coil from a power source.

5. The cryoprobe of claim 4, further comprising: an external electro-insulation case, which protects said electrical heating coil.

6. The cryoprobe of claim 3, wherein a proximal section of said sliding tubular piece is provided with a fixation unit in order to fix location of said sliding tubular piece relative to said external shaft.

7. The cryoprobe of claim 6, wherein the fixation unit comprises a threaded opening in the proximal section of the sliding tubular piece and a screw in said threaded opening.

8. The cryoprobe of claim 3, wherein the electrical heating coil also provides for measuring temperature of the sliding tubular piece.

9. The cryoprobe of claim 3, wherein the displaceable sheath comprises a separate means for temperature measuring.

10. A method for treating a tissue while preventing damage to an upper or superficial layer thereof, comprising: treating a subject with a cryoprobe according to claim 1.
